(19) United States
(12) Patent Application Publication
(10) Pub. No.: US 2022/0354583 A1
(43) Pub. Date: Nov. 10, 2022

(54) SURGICAL MICROSCOPE SYSTEM, CONTROL APPARATUS, AND CONTROL METHOD

(71) Applicant: SONY GROUP CORPORATION, TOKYO (JP)

(72) Inventors: JUNICHIRO ENOKI, TOKYO (JP); YOSHIO SOMA, TOKYO (JP)

(21) Appl. No.: 17/619,973

(22) PCT Filed: Jun. 22, 2020
(86) PCT No.: PCT/JP2020/024328

§ 371 Date: Dec. 16, 2021

(30) Foreign Application Priority Data
Jun. 25, 2019 (JP) 2019-117384

(51) Int. Cl. A61B 34/20; A61B 90/25; A61B 90/00

(52) U.S. Cl. CPC A61B 34/20; A61B 90/25; A61B 90/361; A61B 2034/2055

(57) ABSTRACT

In order to provide a surgical microscope system (100), a control apparatus (120), and a control method that enable position and orientation of a lens tube to be adjusted without requiring a large-scale system, the surgical microscope system includes an arm (112), a surgical microscope (113), a target value setting unit (122), an estimation unit (123), and a control unit (125), the arm includes a rotatable joint (118), the surgical microscope includes a microscope optical system (114) and a camera (115) that captures an operative field image that is a microscope magnification image of an operative field by the microscope optical system, the surgical microscope being supported by the arm, the target value setting unit sets target values of position and orientation of the surgical microscope, the estimation unit estimates the position and orientation of the surgical microscope on the basis of the operative field image and generates estimated values, and the control unit is configured to control a rotation of the joint in accordance with results of comparison of the target values with the estimated values.

### TECHNICAL FIELD

The present technology relates to a surgical microscope system, a control apparatus, and a control method that are associated with surgical microscope operations.

### BACKGROUND ART

Surgical microscopes to be used in various types of surgery include microscopes that are connected to movable arms so that the positions and orientations of the microscopes can be adjusted. The manners to move a microscope include a “coarse movement” in which the user holds the microscope and directly moves the microscope and a “fine movement” in which the user drives the arms through a foot switch or the like.

In this coarse movement, there is a problem in that an object to be observed easily moves out of a field-of-view of the microscope (hereinafter, operative field) due to the coarse movement. Since the surgical microscope is high-powered, the operative field greatly changes due to a small deviation of the angle or position. Moreover, since the coarse movement is performed by checking with eyes, there is also a problem in that it is not easy to correctly set the angle and distance with respect to the object to be observed and it is difficult to perform highly-reproducible observation.

In particular, as for a surgical microscope including a multi-articulated arm capable of free arm movement that realizes observation at various angles and positions, parameters to move increase, and therefore those problems are more remarkable. On the other hand, the surgical microscope is provided with a camera instead of an eyepiece portion of a conventional surgical microscope, and head up surgery (HUS) that is conducted while observing a 3D video through a monitor or a head-mounted display is spreading.

As one of advantages of the HUS, it is possible to realize observation at free position and angle without being affected by the eyepiece portion of the surgical microscope. Therefore, it is predicted that surgical microscopes capable of free arm movement will increase than before along with the spread of the HUS, and cases where such problems during the coarse movement arise will increase.

For adjusting the position and attitude of the surgical microscope, a technology capable of automatic control has also been proposed. For example, Patent Literature 1 has disclosed a system that detects a microscope position and automatically controls the microscope position and a system including a mechanism that takes a wide-angle image and cuts out a captured image by an electronic zoom.

### CITATION LIST

### Patent Literature

Patent Literature 1: Japanese Patent Application Laid-open No. 2018-99297

### DISCLOSURE OF INVENTION

### Technical Problem

As described in Patent Literature 1, the system that automatically controls the microscope position is capable of obtaining a desired operative field while it needs a mechanism that automatically moves the microscope like a robotic arm, which results in a large-scale system. Moreover, the moving microscope can come into contact with the surrounding person or object, and there can be safety and sanitary risks.

In view of the above-mentioned circumstances, it is an object of the present technology to provide a surgical microscope system, a control apparatus, and a control method that enable the position and orientation of the surgical microscope to be adjusted without requiring a large-scale system.

### Solution to Problem

In order to accomplish the above-mentioned object, a surgical microscope system according to the present technology includes an arm, a surgical microscope, a target value setting unit, an estimation unit, and a control unit.

The arm includes a rotatable joint.

The surgical microscope includes a microscope optical system and a camera that captures an operative field image that is a microscope magnification image of an operative field by the microscope optical system, the surgical microscope being supported by the arm.

The target value setting unit sets target values of position and orientation of the surgical microscope.

The estimation unit estimates the position and orientation of the surgical microscope on the basis of the operative field image and generates estimated values.

The control unit controls a rotation of the joint (e.g., imposes a limitation on the rotation) in accordance with results of comparison of the target values with the estimated values.

The control unit may increase a rotational resistance of the joint in a case where the joint rotates in a direction to increase differences between the target values and the estimated values.

The arm may calculate, with respect to a plurality of joints, amounts of change of differences between the target values and the estimated values when the plurality of joints rotates by a certain of amount, and set a rotational resistance of the joint the amount of change of which is larger to be higher than a rotational resistance of the joint the amount of change of which is smaller.

The control unit may set a rotational resistance of the joint to become higher as differences between the target values and the estimated values become larger.

The control unit may stop the rotation of the joint in a case where the joint rotates in a direction to increase differences between the target values and the estimated values.

The control unit may stop the rotation of the joint in a case where the joint is at an angle of rotation that minimizes differences between the target values and the estimated values.

The surgical microscope system may further include a degree-of-adaptability calculation unit that compares the target values with the estimated values and calculates degrees of adaptability of the target values and the estimated values, and the control unit may impose a limitation on the rotation of the joint in accordance with the degrees of adaptability.

The control unit may further impose a limitation on the rotation of the joint in accordance with a distance at which the camera is capable of imaging.

The surgical microscope system may further include a standard object determination unit that determines a standard object on the basis of the operative field image captured by the camera and the target value setting unit may set the position and orientation of the surgical microscope with respect to a position of the standard object as the target values.

The target value setting unit may set the position and orientation of the surgical microscope with respect to a position of an object to be imaged included in the operative field image captured by the camera as the target values.

The target value setting unit may retain the position and orientation of the surgical microscope in past as presets of the target values and set the target values by user's selection from the presets.

The estimation unit may utilize an internal parameter of the camera for estimating the position and orientation of the surgical microscope, and the control unit may change the microscope optical system to have such a setting that the internal parameter retained by the estimation unit is capable of being utilized.

In order to accomplish the above-mentioned object, a control apparatus according to the present technology is a control apparatus for a surgical microscope including a microscope optical system and a camera that captures an operative field image that is a microscope magnification image of an operative field by the microscope optical system, the surgical microscope being supported by an arm including a rotatable joint.

The control apparatus includes a target value setting unit, an estimation unit, and a control unit.

The target value setting unit sets target values of position and orientation of the surgical microscope.

The estimation unit estimates the position and orientation of the surgical microscope on the basis of the operative field image and generates estimated values.

The control unit that controls a rotation of the joint in accordance with results of comparison of the target values with the estimated values.

The control unit controls a rotation of the joint (e.g., imposes a limitation on the rotation) in accordance with results of comparison of the target values with the estimated values.

In order to accomplish the above-mentioned object, a control method according to the present technology is a control method for a surgical microscope including a microscope optical system and a camera that captures an operative field image using the microscope optical system, the surgical microscope being supported by an arm including a joint, the control method including: setting, by a target value setting unit, target values of position and orientation of the surgical microscope; estimating, by an estimation unit, the position and orientation of the surgical microscope on the basis of the operative field image and generating estimated values; and controlling, by a control unit, a rotation of the joint (e.g., imposing a limitation on the rotation) in accordance with results of comparison of the target values with the estimated values.

### BRIEF DESCRIPTION OF DRAWINGS

FIG. 1 A block diagram showing a configuration of a surgical microscope system according to an embodiment of the present technology.

FIG. 2 A schematic view of a surgical microscope unit of the surgical microscope system.

FIG. 3 A schematic view showing an operation of the surgical microscope unit.

FIG. 4 A flowchart of a first processing flow of the surgical microscope system.

FIG. 5 A schematic view showing a movement of a joint of an arm of the surgical microscope unit.

FIG. 6 A schematic view showing UI display processing by the surgical microscope system.

FIG. 7 A flowchart of a second processing flow of the surgical microscope system.

FIG. 8 A schematic view showing an estimation method for distance and angle of a camera with respect to an object to be imaged in the surgical microscope system.

FIG. 9 A block diagram showing hardware configurations of a control apparatus of the surgical microscope system.

### MODE(S) FOR CARRYING OUT THE INVENTION

A surgical microscope system according to an embodiment of the present technology will be described.

[Configuration of Surgical Microscope System]

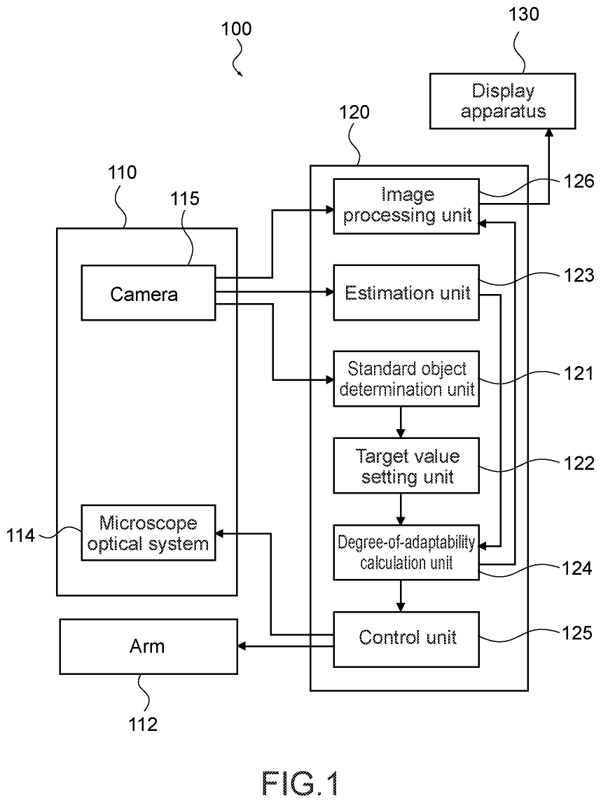

FIG. 1 is a block diagram showing a configuration of a surgical microscope system 100 according to this embodiment. As shown in the figure, the surgical microscope system 100 includes a surgical microscope unit 110, a control apparatus 120, and a display apparatus 130.

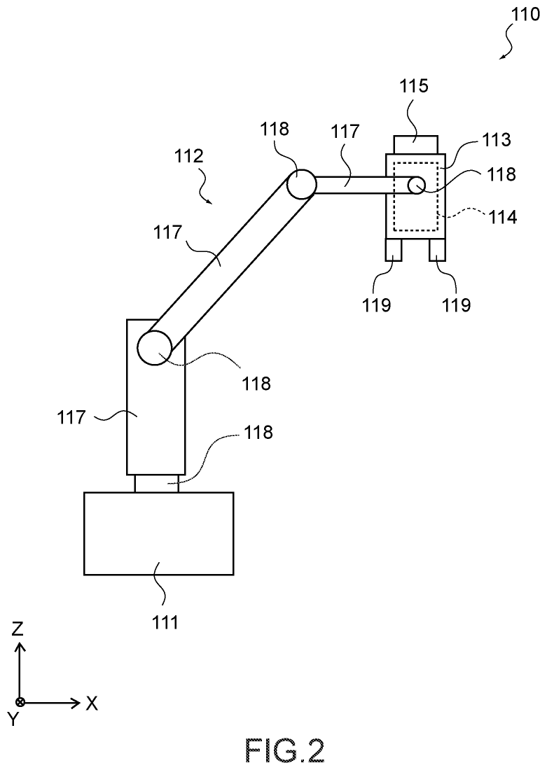

FIG. 2 is a schematic view of the surgical microscope unit 110. As shown in the figure, the surgical microscope unit 110 includes a base 111, an arm 112, a surgical microscope 113, a microscope optical system 114, and a camera 115.

The arm 112 is movable and supports the surgical microscope 113. As shown in FIG. 2, the arm 112 includes a plurality of arm portions 117 and a plurality of joints 118. Each joint 118 connects the adjacent arm portions 117 to each other and is rotatable. Due to a rotation of each joint 118, each arm portion 117 changes in angle with respect to the adjacent arm portion 117 and the surgical microscope 113 can be put in arbitrary position and orientation.

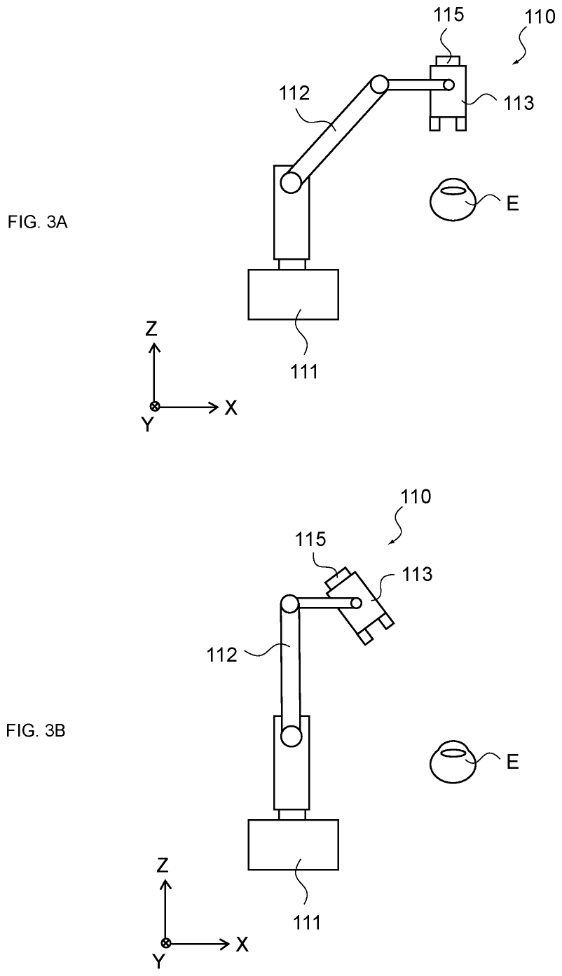

FIG. 3 is a schematic view showing a movement of the arm 112 and shows changes in the position and orientation of the surgical microscope 113 with respect to an object to be observed. In the figure, an eyeball E is shown as an example of the object to be observed. As shown in FIGS. 3 (a) and (b), the surgical microscope 113 is movable in each direction due to a movement of the arm 112 and the surgical microscope 113 can be tilted in each direction.

It should be noted that a specific configuration of the arm 112 is not limited to one shown in FIGS. 2 and 3, and it is sufficient that the arm 112 includes at least one arm portion 117 and at least one joint 118 and is capable of changing the position and orientation of the surgical microscope 113.

The surgical microscope 113 is supported by the arm 112 and is configured to be capable of changing the position and orientation. The surgical microscope 113 includes the microscope optical system 114 including an objective lens 119 and other optical elements (not shown).

The camera 115 is provided in the surgical microscope 113 and captures a microscope magnification image generated by the microscope optical system 114. The camera 115 only needs to be capable of capturing a still image or a moving image of the microscope magnification image. It should be noted that in the following description, a field-of-view range imaged by the camera 115 via the microscope optical system 114 will be referred to as an “operative field” and the image or moving image obtained by imaging the operative field through the camera 115 will be referred to as an “operative field image”. The camera 115 outputs the operative field image to the control apparatus 120.

The surgical microscope unit 110 is configured to be capable of coarse movement. The coarse movement is a movement of the arm 112 by the user, and the user can hold the surgical microscope 113 and move the surgical microscope 113 in an arbitrary direction. Moreover, the arm 112 may be capable of fine movement in addition to the coarse movement. The fine movement is a movement of the arm 112 by the arm 112 itself, and the arm 112 is moved by rotationally driving each joint 118. The user can instruct the surgical microscope unit 110 to perform a fine movement by using a foot switch or the like.

As shown in FIG. 1, the control apparatus 120 includes a standard object determination unit 121, a target value setting unit 122, an estimation unit 123, a degree-of-adaptability calculation unit 124, a control unit 125, and an image processing unit 126. The respective configurations of the control apparatus 120 are functional configurations that are realized by cooperation of software with hardware. The control apparatus 120 may be an information processing apparatus independent of the surgical microscope unit 110 or may be installed in a camera control unit (CCU) of the surgical microscope unit 110. Alternatively, the control apparatus 120 may be connected to the surgical microscope unit 110 via a network.

Hereinafter, the respective configurations of the control apparatus 120 will be described, and the details will be described with an operation of the surgical microscope system 100 to be described later.

The standard object determination unit 121 acquires an operative field image from the camera 115 and detects and determines an object that is a standard (hereinafter, standard object). The standard object may be the entire operative field shown in the operative field image or may be a partial region of the operative field image, a surgical instrument or a biological tissue (e.g., eyeball). The standard object determination unit 121 supplies the determined standard object to the target value setting unit 122.

The target value setting unit 122 sets “target values”. The target values are the position and orientation of the surgical microscope 113 after a coarse movement when the user moves the arm 112 through the coarse movement. The target value setting unit 122 is capable of setting target values by the user's designation or selection from presets. The target value setting unit 122 supplies the set target values to the degree-of-adaptability calculation unit 124.

The estimation unit 123 estimates the position and orientation of the surgical microscope 113 on the basis of the operative field image. Hereinafter, the position and orientation of the surgical microscope 113 that are estimated by the estimation unit 123 will be referred to as “estimated values”. The estimation unit 123 is capable of generating estimated values by a simultaneous localization and mapping (SLAM) technology or the like. The estimation unit 123 supplies the generated estimated values to the degree-of-adaptability calculation unit 124.

The degree-of-adaptability calculation unit 124 compares the target values supplied from the target value setting unit 122 with the estimated values supplied from the estimation unit 123 and calculates degrees of adaptability. The degree-of-adaptability calculation unit 124 is capable of calculating a degree of adaptability for each of parameters (distance and orientation). The degree-of-adaptability calculation unit 124 supplies the calculated degrees of adaptability to the control unit 125 and the image processing unit 126.

The control unit 125 controls the movement of the arm 112 in accordance with results of comparison of the target values with the estimated values and specifically, controls the movement of the arm 112 on the basis of the degrees of adaptability supplied from the degree-of-adaptability calculation unit 124. Specific contents of the arm control will be described later. Moreover, the control unit 125 is favorably capable of controlling a zoom scale, a diaphragm size, or the like of the microscope optical system 114.

The image processing unit 126 generates a display image from the operative field image captured by the camera 115 and causes the display apparatus 130 to display the generated display image. At this time, the image processing unit 126 may perform user interface (UI) display processing in accordance with the degrees of adaptability supplied from the degree-of-adaptability calculation unit 124.

The display apparatus 130 displays the display image output from the image processing unit 126. The display apparatus 130 may be a general display or may be a three-dimensional display or a head-mounted display capable of displaying a 3D video.

[Operation of Surgical Microscope System]

A coarse movement operation of the surgical microscope system 100 will be described. A use case where the coarse movement of the surgical microscope unit 110 is used in the surgical microscope system 100 includes the following two cases.

The first case is a case where there is a demand for starting a coarse movement from a state in which a standard object is present in the operative field and performing observation by changing at least one of the angle or distance of the surgical microscope 113 with respect to the standard object. For example, as shown in FIG. 3 (a), there can be a case where in ophthalmic surgery, after an incision is made in a state in which the surgical microscope 113 is placed in a vertical direction of the eyeball E, the surgical microscope 113 is tilted and eye-corner treatment is performed obliquely to the eyeball E as shown in FIG. 3 (b).

The second case is a case of starting a coarse movement from a state in which the standard object is absent in the operative field and obtaining a first operative field. For example, there can be a case of performing setting up for obtaining the first operative field at the time of starting surgery.

Hereinafter, a processing flow of the surgical microscope system 100 will be described with respect to each of the two cases.

(Processing Flow with Respect to First Case)

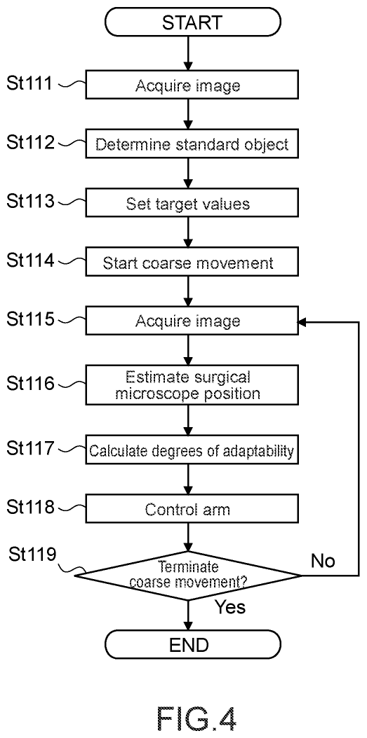

The surgical microscope system 100 performs a processing flow with respect to the first case (hereinafter, first processing flow), receiving a user's instruction. The user is able to instruct the surgical microscope system 100 to start the processing flow through a physical switch or touch panel provided in the surgical microscope 113, a foot switch, an audio command, or the like. FIG. 4 is a flowchart showing the first processing flow.

When the user's instruction is input, the standard object determination unit 121 acquires an operative field image from the camera 115 (St111). The standard object determination unit 121 may acquire a still image. Moreover, the camera 115 continues to perform imaging during the surgery, and therefore some latest frames may be constantly recorded and the standard object determination unit 121 may extract an image from them. At this time, it is favorable that the standard object determination unit 121 extracts an image that is as closer as possible to the timing when the user's instruction is input and that is less affected by shaking, less blurred, and less covered.

The standard object determination unit 121 determines a standard object from the acquired operative field image (St112). The standard object may be the entire operative field shown in the operative field image or may be a region of the operative field image, the operative field center portion or focus of which is appropriately adjusted. Alternatively, the standard object determination unit 121 may set a site of a living body which is designated from the user or an object such as a surgical instrument as the standard object. The user is able to refer to the operative field image through the display apparatus 130 and to designate an object to be the standard object.

Moreover, the standard object determination unit 121 may determine the standard object by an image recognition technology. For example, when the user inputs the name of an object such as an “eyeball” and a “gonioscope”, the standard object determination unit 121 is capable of detecting such an object by the image recognition technology and determining it as the standard object.

In addition, the standard object determination unit 121 may determine the standard object by extracting an identical site including a point pointed by the user by region detection. For example, when the user points a part of the eye, the standard object determination unit 121 is capable of determining the entire eyeball as the standard object. At this time, the standard object determination unit 121 is capable of using a technology of a machine learning system or a technology based on feature point extraction as the image recognition technology. Alternatively, the standard object determination unit 121 may detect a maker added to a surgical instrument or the like, detect the surgical instrument or the like, and set it as the standard object.

The target value setting unit 122 sets the position and orientation of the surgical microscope 113 after a coarse movement with respect to the position of the standard object (hereinafter, standard position) as “target values” (St113). The target value setting unit 122 is cable of setting the target values by the user's designation. The user is able to designate the target values as the distance and orientation of the object to be observed using the standard position as the point-of-origin, and designates the target values, for example, as “angle of 45 degrees, working distance (WD)=150 mm” with respect to the standard position. The user may designate both of the distance and orientation as the target values or may designate only one of the distance and orientation as the target value.

Moreover, the target value setting unit 122 may include presets of the target values in advance. By selecting target values from the presets, the user can omit the time and effort to set the same values when repeating similar arm movements. The target value setting unit 122 can include presets depending on the type of surgery, such as cataract surgery and glaucoma surgery, for example.

In addition, there is a case where after the user moves the arm 112 once, the user wishes to restore the state before the movement. Therefore, the target value setting unit 122 may store the position and orientation of the surgical microscope 113 in the past as presets so that the user can select.

Subsequently, the user moves the arm 112 and starts a coarse movement (St114). It should be noted that the above-mentioned steps (S111 to St113) may be performed while the user performs the coarse movement.

Subsequently, the estimation unit 123 acquires an operative field image from the camera 115 (St115). The estimation unit 123 is capable of regularly acquiring operative field images while the coarse movement is performed. Acquisition timings for the operative field images may be on a frame basis or may be at constant intervals.

Subsequently, the estimation unit 123 estimates the position and orientation of the surgical microscope 113 with respect to the standard position on the basis of the acquired operative field image (St116) and generates estimated values. The estimation unit 123 is capable of generating the estimated values by a simultaneous localization and mapping (SLAM) technology. Since generation of an environment map is also performed in the SLAM, there is an advantage that the standard object does not need to be shown in the operative field during the coarse movement.

Alternatively, the estimation unit 123 may generate the estimated values by comparison of feature points of the operative field image. The user is able to perform the coarse movement while viewing the operative field, and therefore the standard object can be controlled to be shown in the operative field to some extent. Therefore, the estimated values can also be generated by determining the correspondence of feature points included in the image in the acquired operative field image and solving a Perspective-n-Point Problem in the two image acquisition steps (St111 and St115).

Moreover, in a case where the operative field image is a stereo image, the estimation unit 123 is capable of generating the estimated values by generating a depth map on the basis of the operative field image. Furthermore, the estimation unit 123 may generate the estimated values by a time-of-flight (TOF) technology.

Subsequently, the degree-of-adaptability calculation unit 124 compares the target values set in the target value setting step (St113) with the estimated values generated in the surgical microscope position estimating step (St116) and calculates degrees of adaptability (St117). The degree-of-adaptability calculation unit 124 is capable of calculating a degree of adaptability for each of parameters (distance and orientation), and for example, calculates degrees of adaptability as “degree of adaptability of distance: 90%, degree of adaptability of angle: 85%”.

Subsequently, the control unit 125 controls the movement of the arm 112 (St118). Specifically, the control unit 125 imposes a limitation on rotations of the joints 118 on the basis of the degrees of adaptability calculated in the degree-of-adaptability calculation step (St117).

The control unit 125 calculates, for each joint 118, amounts of change of the degrees of adaptability when the joint 118 rotates by a certain amount. When the user moves the surgical microscope 113 in order to change at least one of the position or the orientation of the surgical microscope 113, the control unit 125 determines whether each joint 118 rotates in a direction to decrease the degrees of adaptability or rotates in a direction to increase the degrees of adaptability.

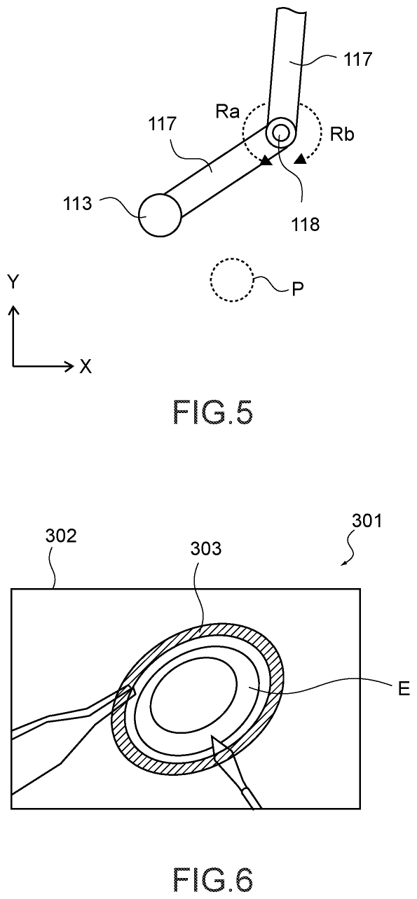

FIG. 5 is a schematic view showing an example of the rotation of the joint 118. In the figure, the target values set by the target value setting unit 122 are shown as target values P. The position and orientation of the surgical microscope 113 have been estimated by the estimation unit 123 and supplied as the estimated values to the control unit 125.

In the figure, a direction of rotation Ra of the joint 118 is a direction of rotation in which the surgical microscope 113 approaches the target values P, and therefore it is a direction of rotation to increase the degrees of adaptability. On the other hand, a direction of rotation Rb is a direction of rotation in which the surgical microscope 113 moves away from the target values P, and therefore it is a direction of rotation to decrease the degrees of adaptability. The control unit 125 determines, for each joint 118, the direction of rotation in which the degrees of adaptability increase and the direction of rotation in which the degrees of adaptability decrease. Moreover, the control unit 125 calculates amounts of change of the degrees of adaptability which are associated with the rotation amount of the joint 118. In FIG. 5, the movement of the surgical microscope 113 in an X-Y direction is shown, though the movement of the surgical microscope 113 in a Z direction is performed similarly.

In a case where the joint 118 rotates in a direction in which the differences between the target values and the estimated values increase, that is, the direction to decrease the degrees of adaptability, the control unit 125 is capable of braking the joint 118 and increasing the rotational resistance. In the example shown in FIG. 5, the rotational resistance of the direction of rotation Rb is made higher than the rotational resistance of the direction of rotation Ra. Moreover, when the surgical microscope 113 passes through the target values P, the direction of rotation Ra is the direction of rotation to decrease the degrees of adaptability and the control unit 125 increases the rotational resistance of the direction of rotation Ra.

Accordingly, when the user attempts to move the surgical microscope 113 in the direction to decrease the degrees of adaptability, the force required for moving the surgical microscope 113 increases, and therefore it becomes easy to adjust the surgical microscope 113 to the target values.

Moreover, the control unit 125 may change the rotational resistance for each joint 118. The control unit 125 sets the rotational resistance of the joints 118 for which the amounts of change of the differences between the target values and the estimated values when it rotates by the certain amount are larger, that is, the amounts of decrease of the degrees of adaptability are larger, to be higher than the rotational resistance of the joints 118 for which such amounts of change are smaller, that is, the amounts of decrease of the degrees of adaptability are smaller. The joint 118 whose amounts of change are larger is the joint 118 having a larger rotation amount associated with the movement of the surgical microscope 113 and the joint 118 whose amounts of change are smaller is the joint 118 having a smaller rotation amount associated with the movement of the surgical microscope 113. Also in this case, when the user attempts to move the surgical microscope 113 in the direction in which the degrees of adaptability decrease, the force required for moving the surgical microscope 113 increases, and therefore it becomes easy to adjust the surgical microscope 113 to the target values.

Furthermore, the control unit 125 may increase the rotational resistance of the joints 118 as the differences between the target values and the estimated values become larger, that is, the degrees of adaptability become lower. Accordingly, when the position of the surgical microscope 113 has moved away from the target values, it is easy to move the surgical microscope 113, and when the position of the surgical microscope 113 has approached the target values, it is difficult to move the surgical microscope 113, and therefore it becomes easy to adjust the surgical microscope 113 to the target values.

Moreover, when the joint 118 rotates a direction in which the differences between the target values and the estimated values increase, that is, the direction to decrease the degrees of adaptability, the control unit 125 may lock the joint 118 and stop the rotation. In the example shown in FIG. 5, the rotation in the direction of rotation Ra is allowed and the rotation of the direction of rotation Rb is stopped. Accordingly, the user is enabled to move the surgical microscope 113 only in the direction to increase the degrees of adaptability, and therefore it becomes easy to adjust the surgical microscope 113 to the target values.

Moreover, the control unit 125 calculates degrees of adaptability for each angle of rotation of each joint 118 and each joint 118 may determine an angle of rotation that minimizes the differences between the target values and the estimated values, that is, an angle of rotation that maximizes the degrees of adaptability. The control unit 125 is capable of stopping the rotation of the joint 118 having the angle of rotation that minimizes the differences between the target values and the estimated values. Accordingly, the rotations of the joints 118 are sequentially fixed in the order from the joint 118 adjusted to have the angle of rotation that minimizes the differences between the target values and the estimated values, and it becomes easy to adjust the surgical microscope 113 to the target values.

Moreover, the control unit 125 may limit the rotations of the joints 118 in accordance with a distance in which the camera 115 is capable of imaging. For example, in a case where the camera 115 has an autofocus function, a focused operative field image cannot be captured when the surgical microscope 113 is located outside the range of autofocus.

Therefore, when the surgical microscope 113 is likely to be out of the range in which the autofocus is valid, the control unit 125 is capable of locking the rotation of the joint 118 that moves the surgical microscope 113 in that direction. In addition to the autofocus range, the control unit 125 is capable of limiting the rotation of the joint 118 on the basis of the range in which the camera 115 is capable of imaging in a manner that depends on optical characteristics and the like of the microscope optical system 114.

In a case where the coarse movement is continued (St119: No), the surgical microscope system 100 repeatedly performs the image acquisition step (St115) to the arm control step (St118), and terminates the processing flow when the coarse movement is finished (St119: Yes).

It should be noted that in the step (St118) of controlling the movement of the arm 112, user interface (UI) display processing according to the degrees of adaptability may be performed in addition to the arm movement control.

FIG. 6 is a schematic view showing a display image 301 as an example of the display image generated by the image processing unit 126. As shown in the figure, the image processing unit 126 is capable of arranging an indicator 303 indicating degrees of adaptability in the periphery or the like of the eyeball E in an operative field image 302 and generating the display image 301. The indicator 303 changes the color, the shape, the transparency, or the like in accordance with the degrees of adaptability and presents the degrees of adaptability to the user.

Moreover, the image processing unit 126 may superimpose a character string representing the degrees of adaptability on the operative field image 302 and generate the display image 301 in addition to or instead of the indicator 303. Moreover, instead of the degrees of adaptability, the image processing unit 126 may display angle and distance information of the surgical microscope 113 or may display a degree of deviation from the target values of the surgical microscope 113. Also, in addition to this, the image processing unit 126 is capable of superimposing various types of indication representing differences between the target values and the estimated values on the operative field image 302 and generating the display image 301.

The image processing unit 126 outputs the generated display image to the display apparatus 130. Accordingly, in addition to controlling the rotations of the joints 118, the user is able to refer to the information presented in the display image and move the surgical microscope 113 and it becomes easy to adjust the surgical microscope 113 to be in desired position and orientation.

After the coarse movement is performed following the processing flow, the user may operate the foot switch or the like and move (finely move) the arm 112. At this time, the image processing unit 126 is capable of performing UI display processing according to the degrees of adaptability and displaying the generated display image (see FIG. 6) in the display apparatus 130. Accordingly, the user is capable of referring to the degrees of adaptability also in the fine movement and finely adjusting the position and orientation of the surgical microscope 113.

### Modified Example of First Processing Flow

In the above description, in order to determine the standard object, the operative field image is acquired in the operative field image acquisition step (St111). However, in a case where a particular surgical instrument is set as the standard object, acquiring information regarding the surgical instrument in advance makes the operative field image acquisition step (St111) unnecessary.

For example, in a case where the standard object is a gonioscope, the position and orientation of the surgical microscope 113 can be estimated by detecting the ellipse of the lens edge from the operative field image and acquiring a long-side axis and a short-side axis in the surgical microscope position estimating step (St116).

In addition, in the surgical microscope position estimating step (St116), the following operation may be performed for improving the performance. In order for the estimation unit 123 to estimate the position and orientation of the surgical microscope 113, it is desirable that the internal parameter of the camera be known. The internal parameter of the camera is a transform matrix when transforming three-dimensional coordinates of a camera coordinate point-of-origin into coordinates in the image and is required when estimating a three-dimensional position of the camera 115 on the basis of the operative field image.

However, the internal parameter changes depending on settings of the zoom scale and the like, and therefore it is necessary to retain numerous internal parameters or to replace them by a closer internal parameter in order to cope with it through an arbitrary scale.

In view of this, when a coarse movement is performed, the control unit 125 is capable of controlling the microscope optical system 114 to change the zoom scale, the diaphragm, and the like of the surgical microscope 113 into such settings that the internal parameter retained by the estimation unit 123 can be utilized. Accordingly, the estimation unit 123 is capable of estimating the position and orientation of the surgical microscope 113 by using a known internal parameter, and the estimation accuracy can be improved.

Moreover, if the scale of the microscope optical system is too high, the standard object is more likely to be out of the operative field, and therefore the control unit 125 may control the microscope optical system 114 to have a wider angle to some extent. In addition, it is difficult to estimate the position and orientation of the surgical microscope 113 with an unfocused and blurred image, and therefore the control unit 125 may control the microscope optical system 114 to switch the settings to a mode for a coarse movement, for example, to activate autofocus or reduce the diaphragm so as to increase the depth of field.

(Processing Flow with Respect to Second Case)

The processing flow with respect to the second case (hereinafter, second processing flow) is different from the first processing flow in that the operative field image acquisition step and the standard object determination step at the beginning are not performed in the second processing flow.

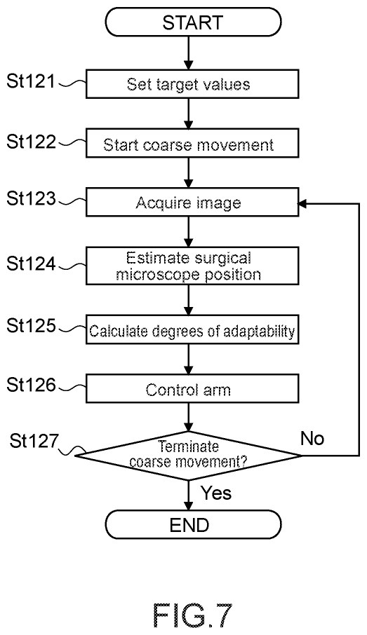

Also in the second processing flow, the surgical microscope system 100 performs the processing flow, receiving a user's instruction. The user is able to instruct the surgical microscope system 100 to start the processing flow through a physical switch or touch panel provided in the surgical microscope 113, a foot switch, an audio command, or the like. FIG. 7 is a flowchart showing the second processing flow.

The target value setting unit 122 sets the position and orientation of the surgical microscope 113 after a coarse movement as “target values” (St121). The target value setting unit 122 is capable of setting the target values by the user's designation. In the second processing flow, the standard object that defines a coordinate system does not exist, and therefore the user is able to designate the target values as a distance from an object captured by the camera 115 (hereinafter, object to be imaged) during the coarse movement and a tilt angle of the surgical microscope 113 or the like. The tilt angle can be a “degree of tilt with respect to a vertical direction in a case where the surface of the object to be imaged is approximated by a plane”. The user is able to designate the target values, for example, as “angle of 45 degrees, working distance (WD)=150 mm” with respect to the standard position. The user may designate both of the distance and orientation as the target values or may designate only one of the distance and orientation.

Moreover, the target value setting unit 122 may include presets of the target values in advance. By selecting target values from the presets, the user can omit the time and effort to set the same values when repeating similar arm movements. The target value setting unit 122 can include target values depending on the type of surgery, such as cataract surgery and glaucoma surgery, for example. For example, the preset for the cataract can be “WD=200 mm vertically with respect to the eyeball”, and the preset for the glaucoma can be “45 degrees, WD=200 mm with respect to the eyeball”, or the like.

In addition, there is a case where after the user moves the arm 112 once, the user wishes to restore the state before the movement. Therefore, the target value setting unit 122 may store the position and orientation of the surgical microscope 113 in the past as presets such that the user can select.

Subsequently, the user moves the arm 112 and starts a coarse movement (St122). It should be noted that the target value setting step (St121) may be performed while the user performs the coarse movement.

Subsequently, the estimation unit 123 acquires an operative field image from the camera 115 (St123). The estimation unit 123 is capable of regularly acquiring operative field images while the coarse movement is performed. Acquisition timings for the operative field images may be on a frame basis or may be at constant intervals.

Subsequently, the estimation unit 123 estimates the position and orientation of the surgical microscope 113 on the basis of the acquired operative field image (St124) and generates estimated values. Only an estimation method that does not require the standard object can be utilized in the second processing flow. Specifically, the estimation unit 123 is capable of generating the estimated values by a simultaneous localization and mapping (SLAM) technology. Since generation of an environment map is also performed in the SLAM, the surface shape of the object to be imaged can also be acquired in addition to the distance from the object to be imaged.

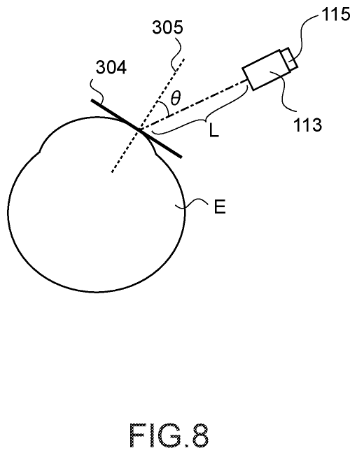

Moreover, in a case where the operative field image is a stereo image, the estimation unit 123 is also capable of determining the distance from the object to be imaged and the surface shape by generating a depth map on the basis of the operative field image. FIG. 8 is a schematic view showing the tilt and the distance with respect to the object to be imaged of the surgical microscope 113. As shown in the figure, when the surface shape of the object to be imaged (here, the eyeball E as an example) is determined, a tilt θ and a distance L of the surgical microscope 113 can be calculated by applying a plane 304 to the surface shape and determining a direction 305 perpendicular to the plane 304.

Subsequently, the degree-of-adaptability calculation unit 124 compares the target values set in the target value setting step (St121) with the estimated values generated in the surgical microscope position estimating step (St124) and calculates degrees of adaptability (St125). The degree-of-adaptability calculation unit 124 is capable of calculating a degree of adaptability for each of parameters (distance and orientation) as in the first processing flow.

Subsequently, the control unit 125 controls the movement of the arm 112 (St126). Specifically, the control unit 125 imposes a limitation on rotations of the joints 118 as in the first processing flow on the basis of the degrees of adaptability calculated in the degree-of-adaptability calculation step (St125). In addition, the control unit 125 may limit the rotations of the joints 118 in accordance with a distance in which the camera 115 is capable of imaging.

In a case where the coarse movement is continued (St127: No), the surgical microscope system 100 repeatedly performs the image acquisition step (St123) to the arm control step (St126), and terminates the processing flow when the coarse movement is finished (St127: Yes).

It should be noted that in the step (St126) of controlling the movement of the arm 112, the image processing unit 126 may perform UI display processing according to the degrees of adaptability (see FIG. 6) as in the first processing flow in addition to the limitation on the rotations of the joints 118, and may support the user's coarse movement.

After the coarse movement is performed following the processing flow, the user may operate the foot switch or the like and move (finely move) the arm 112. Also at this time, the image processing unit 126 is capable of performing UI display processing according to the degrees of adaptability UI (see FIG. 6) and supporting the user's fine movement.

### Modified Example of Second Processing Flow

Also in the second processing flow, when a coarse movement is performed, the control unit 125 is capable of controlling the microscope optical system 114 to change the zoom scale, the diaphragm, and the like of the surgical microscope 113 into such settings that the internal parameter retained by the estimation unit 123 can be utilized. Accordingly, the estimation unit 123 is capable of estimating the position and orientation of the surgical microscope 113 by using a known internal parameter, and the estimation accuracy can be improved.

Moreover, if the scale of the microscope optical system is too high, the standard object is more likely to be out of the operative field, and therefore the control unit 125 may control the microscope optical system 114 to have a wider angle to some extent. In addition, it is difficult to estimate the position and orientation of the surgical microscope 113 with an unfocused and blurred image, and therefore the control unit 125 may control the microscope optical system 114 to switch the settings to a mode for a coarse movement, for example, to activate autofocus or reduce the diaphragm so as to increase the depth of field.

[Effects of Surgical Microscope System]

As described above, in the surgical microscope system 100, the rotations of the joints 118 are limited in accordance with the degrees of adaptability of the target values and the estimated values, and it is possible to adjust the surgical microscope 113 to be in desired position and orientation through a coarse movement.

Accordingly, it becomes easy for the user to observe the object to be observed on the basis of desired distance and angle. Moreover, it is possible to perform highly-reproducible setting by storing the target values in the surgical microscope system 100. In addition, the surgical microscope 113 can be prevented from moving to a position inappropriate for imaging, such as the outside of the autofocus range of the camera 115.

In addition, since the user moves the surgical microscope 113, the surgical microscope system 100 does not require position control by a robotic arm and the like, and a large-scale system is unnecessary. Thus, cost reduction is also possible.

[Hardware Configurations]

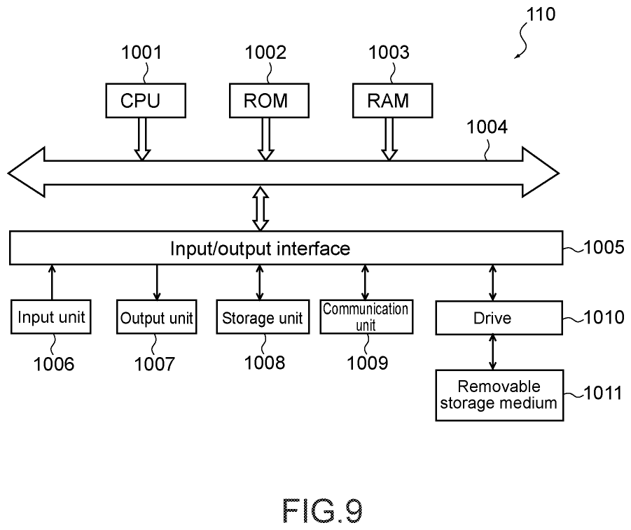

Hardware configurations of the control apparatus 120 will be described. FIG. 9 is a schematic view showing the hardware configurations of the control apparatus 120.

As shown in the figure, the control apparatus 120 includes a built-in central processing unit (CPU) 1001. An input/output interface 1005 is connected to the CPU 1001 via a bus 1004. A read only memory (ROM) 1002 and a random access memory (RAM) 1003 are connected to the bus 1004.

To the input/output interface 1005, are connected an input unit 1006 including input devices such as a keyboard and a mouse for the user to input an operation command, an output unit 1007 that outputs a processing operation screen and a processing result image to a display device, a storage unit 1008 including a hard disk drive that stores programs and various types of data, and a communication unit 1009 that includes a local area network (LAN) adaptor and the like and performs communication processing via a network represented by the Internet. Also, a drive 1010 that reads and writes data from/to a removable storage medium 1011 that is a magnetic disk, an optical disc, a magneto-optical disk, a semiconductor memory, or the like is connected.

The CPU 1001 performs various types of processing in accordance with a program stored in the ROM 1002 or a program that is read from the removable storage medium 1011 that is the magnetic disk, the optical disc, the magneto-optical disk, the semiconductor memory, or the like, installed in the storage unit 1008, and loaded into the RAM 1003 from the storage unit 1008. Data and the like necessary for the CPU 1001 to perform various types of processing are also stored in the RAM 1003 as appropriate. It should be noted that the CPU 1001 may include a graphics processing unit (GPU) function or the control apparatus 120 may include a GPU other than the CPU 1001.

In the control apparatus 120 configured in the above-mentioned manner, the above-mentioned series of processing are performed by, for example, the CPU 1001 loading the program stored in the storage unit 1008 into the RAM 1003 via the input/output interface 1005 and the bus 1004 and executing it.

The program to be executed by the control apparatus 120 can be provided by recording it in the removable storage medium 1011 serving as a package medium or the like, for example. Moreover, the program can be provided via a wired or wireless transmission medium such as a local area network, the Internet, and digital satellite broadcasting.

In the control apparatus 120, the program can be installed in the storage unit 1008 via the input/output interface 1005 by mounting the removable storage medium 1011 on the drive 1010. Moreover, the program can be received by the communication unit 1009 and installed in the storage unit 1008 via the wired or wireless transmission medium. Otherwise, the program can be installed in the ROM 1002 or the storage unit 1008 in advance.

It should be noted that the program to be executed by the control apparatus 120 may be a program whose processes are sequentially performed in the order described in the present disclosure or may be a program whose processes are concurrently performed or at a necessary timing, for example, upon calling.

Moreover, the hardware configurations of the control apparatus 120 do not need to be all installed in a single apparatus, and a plurality of apparatuses may configure the control apparatus 120. Moreover, the hardware configurations of the control apparatus 120 may be installed in a plurality of apparatuses connected via some of the hardware configurations of the control apparatus 120 or the network.

At least two features of the features according to the present technology which have been described above may be combined. That is, the various features described in the respective embodiments may be arbitrarily combined across the respective embodiments. Moreover, the above-mentioned various effects are merely exemplary and not limitative, and further other effects may be provided.

It should be noted that the present technology may also take the following configurations.

(1) A surgical microscope system, including:

an arm including a joint;

a surgical microscope including a microscope optical system and a camera that captures an operative field image by using the microscope optical system, the surgical microscope being supported by the arm; and

a control unit that controls a movement of the arm, in which

the control unit controls a rotation of the joint in accordance with a result of comparison of target values of position and orientation of the surgical microscope with estimated values of the position and orientation of the surgical microscope that are estimated on the basis of the operative field image.

(2) The surgical microscope system according to (1), in which

the control unit increases a rotational resistance of the joint in a case where the joint rotates in a direction to increase differences between the target values and the estimated values.

(3) The surgical microscope system according to (2), in which

the arm includes a plurality of joints, and

the control unit calculates, with respect to the plurality of joints, amounts of change of differences between the target values and the estimated values when the plurality of joints rotates by a certain of amount, and sets a rotational resistance of the joint the amount of change of which is larger to be higher than a rotational resistance of the joint the amount of change of which is smaller.

(4) The surgical microscope system according to any one of (1) to (3), in which

the control unit sets a rotational resistance of the joint to become higher as differences between the target values and the estimated values become larger.

(5) The surgical microscope system according to any one of (1) to (4), in which

the control unit stops the rotation of the joint in a case where the joint increases differences between the target values and the estimated values.

(6) The surgical microscope system according to (5), in which

the control unit stops the rotation of the joint in a case where the joint is at an angle of rotation that minimizes differences between the target values and the estimated values.
(7) The surgical microscope system according to any one of (1) to (6), in which

the control unit compares the target values with the estimated values, calculates degrees of adaptability of the target values and the estimated values, and imposes a limitation on the rotation of the joint in accordance with the degrees of adaptability.

(8) The surgical microscope system according to any one of (1) to (7), in which

the control unit further imposes a limitation on the rotation of the joint in accordance with a distance at which the camera is capable of imaging.

(9) The surgical microscope system according to any one of (1) to (8), in which

the control unit determines a standard object on the basis of the operative field image captured by the camera and sets the position and orientation of the surgical microscope with respect to a position of the standard object as the target values.

(10) The surgical microscope system according to any one of (1) to (8), in which

the control unit sets the position and orientation of the surgical microscope with respect to a position of an object to be imaged included in the operative field image captured by the camera as the target values.

(11) The surgical microscope system according to any one of (1) to (10), in which

the control unit retains the position and orientation of the surgical microscope in past as presets of the target values and sets the target values by user's selection from the presets.

(12) The surgical microscope system according to any one of (1) to (11), in which

the control unit utilizes an internal parameter of the camera for estimating the position and orientation of the surgical microscope, and changes the microscope optical system to have such a setting that the internal parameter retained by the estimation unit is capable of being utilized.

(13) A control apparatus for a surgical microscope including a microscope optical system and a camera that captures an operative field image using the microscope optical system, the surgical microscope being supported by an arm including a joint, including:

a target value setting unit that sets target values of position and orientation of the surgical microscope;

an estimation unit that estimates the position and orientation of the surgical microscope on the basis of the operative field image and generates estimated values; and

a control unit that controls a rotation of the joint in accordance with results of comparison of the target values with the estimated values.

(14) A control method for a surgical microscope including a microscope optical system and a camera that captures an operative field image using the microscope optical system, the surgical microscope being supported by an arm including a joint, including:

setting, by a target value setting unit, target values of position and orientation of the surgical microscope;

estimating, by an estimation unit, the position and orientation of the surgical microscope on the basis of the operative field image and generating estimated values; and

controlling, by a control unit, a rotation of the joint in accordance with results of comparison of the target values with the estimated values.

### REFERENCE SIGNS LIST

100 surgical microscope system
110 surgical microscope unit
112 arm
113 surgical microscope
114 microscope optical system
115 camera
117 arm portion
118 joint
120 control apparatus
121 standard object determination unit
122 target value setting unit
123 estimation unit
124 degree-of-adaptability calculation unit
125 control unit
126 image processing unit
130 display apparatus

1. A surgical microscope system, comprising:
an arm including a joint;
a surgical microscope including a microscope optical system and a camera that captures an operative field image by using the microscope optical system, the surgical microscope being supported by the arm; and
a control unit that controls a movement of the arm, wherein
the control unit controls a rotation of the joint in accordance with a result of comparison of target values of position and orientation of the surgical microscope with estimated values of the position and orientation of the surgical microscope that are estimated on a basis of the operative field image.

2. The surgical microscope system according to claim 1, wherein
the control unit increases a rotational resistance of the joint in a case where the joint rotates in a direction to increase differences between the target values and the estimated values.

3. The surgical microscope system according to claim 2, wherein
the arm includes a plurality of joints, and
the control unit calculates, with respect to the plurality of joints, amounts of change of differences between the target values and the estimated values when the plurality of joints rotates by a certain of amount, and sets a rotational resistance of the joint the amount of change of which is larger to be higher than a rotational resistance of the joint the amount of change of which is smaller.

4. The surgical microscope system according to claim 1, wherein
the control unit sets a rotational resistance of the joint to become higher as differences between the target values and the estimated values become larger.

5. The surgical microscope system according to claim 1, wherein
the control unit stops the rotation of the joint in a case where the joint rotates in a direction to increase differences between the target values and the estimated values.

6. The surgical microscope system according to claim 5, wherein
the control unit stops the rotation of the joint in a case where the joint is at an angle of rotation that minimizes differences between the target values and the estimated values.

7. The surgical microscope system according to claim 1, wherein
the control unit compares the target values with the estimated values, calculates degrees of adaptability of the target values and the estimated values, and imposes a limitation on the rotation of the joint in accordance with the degrees of adaptability.

8. The surgical microscope system according to claim 1, wherein
the control unit further imposes a limitation on the rotation of the joint in accordance with a distance at which the camera is capable of imaging.

9. The surgical microscope system according to claim 1, wherein
the control unit determines a standard object on a basis of the operative field image captured by the camera and sets the position and orientation of the surgical microscope with respect to a position of the standard object as the target values.

10. The surgical microscope system according to claim 1, wherein
the control unit sets the position and orientation of the surgical microscope with respect to a position of an object to be imaged included in the operative field image captured by the camera as the target values.

11. The surgical microscope system according to claim 1, wherein
the control unit retains the position and orientation of the surgical microscope in past as presets of the target values and sets the target values by user's selection from the presets.

12. The surgical microscope system according to claim 1, wherein
the control unit utilizes an internal parameter of the camera for estimating the position and orientation of the surgical microscope, and changes the microscope optical system to have such a setting that the internal parameter retained by the estimation unit is capable of being utilized.

13. A control apparatus for a surgical microscope including a microscope optical system and a camera that captures an operative field image using the microscope optical system, the surgical microscope being supported by an arm including a joint, comprising:
a target value setting unit that sets target values of position and orientation of the surgical microscope;
an estimation unit that estimates the position and orientation of the surgical microscope on a basis of the operative field image and generates estimated values; and
a control unit that controls a rotation of the joint in accordance with results of comparison of the target values with the estimated values.

14. A control method for a surgical microscope including a microscope optical system and a camera that captures an operative field image using the microscope optical system, the surgical microscope being supported by an arm including a joint, comprising:
setting, by a target value setting unit, target values of position and orientation of the surgical microscope;
estimating, by an estimation unit, the position and orientation of the surgical microscope on a basis of the operative field image and generating estimated values; and
controlling, by a control unit, a rotation of the joint in accordance with results of comparison of the target values with the estimated values.
